(19) United States
(12) Patent Application Publication
(10) Pub. No.: US 2001/0051812 A1
(43) Pub. Date: Dec. 13, 2001

(54) Treating instrument for endoscope

(75) Inventor: Teruo Ouchi, Saitama (JP)

Correspondence Address: GREENBLUM & BERNSTEIN, P.L.C., 1941 ROLAND CLARKE PLACE, RESTON, VA, 20191, US

(73) Assignee: ASAHI KOGAKU KOGYO KABUSHIKI KAISHA, Tokyo (JP)

(21) Appl. No.: 09/862,303

(22) Filed: May 23, 2001

(30) Foreign Application Priority Data
May 24, 2000 (JP) P2000-152525

(51) Int. Cl. A61B 17/28

(52) U.S. Cl. 606/205

(57) ABSTRACT

An endoscopic treating instrument has a flexible sheath made of a super-elastic alloy pipe.

### BACKGROUND OF THE INVENTION

The present invention relates to a treating instrument for an endoscope for use in insertion in a treating instrument-pass channel of the endoscope.

In most cases, a treating instrument for an endoscope has a structure in which operation wires for operating a forward end operation portion attached to a forward end of a flexible sheath are inserted and disposed in the flexible sheath so as to be movable back and forth axially. As the flexible sheath, a flexible tube such as a coiled pipe of stainless steel helically wound with a predetermined diameter or an ethylene tetrafluoride resin tube is used.

The coiled pipe is, however, apt to be insanitary because dirt is apt to remain the gaps between adjacent wires and because it is impossible to clean such dirt easily even in the case where a cleaning solution is poured.

On the other hand, the flexible tube have to be thickened to a certain degree or more because the flexible tube is weak in knead. Hence, the outer diameter of the flexible tube becomes thick. Moreover, the direction of the forward end portion of the flexible tube is not stabilized when the forward end portion of the flexible tube is protruded from the endoscope because the flexible tube is apt to get into the habit of bending. There is some case where it is difficult to lead the forward end portion of the flexible tube to a target position.

### SUMMARY OF THE INVENTION

Therefore, an object of the present invention is to provide a treating instrument for an endoscope in which: dirt is hardly deposited on a flexible sheath; the flexible tube can be cleaned easily; the outer diameter of the flexible sheath can be formed to be small; and the flexible sheath hardly gets into the habit of bending.

To achieve the foregoing object, a treating instrument for an endoscope according to the present invention comprises a flexible sheath, and operation wires inserted and disposed in the flexible sheath so as to be movable back and forth axially, wherein the flexible sheath is a pipe made of a super-elastic alloy.

The term “super-elastic” means such a characteristic that if an external stress is applied, the deformation of a large strain region (several percents or several tens percents) exceeding a normal elastic strain is caused in accordance with martensitic transformation, and if the external stress is removed, this deformation disappears completely or almost completely in accordance with reverse transformation. The term “super-elastic alloy” means an alloy that exhibits excellent elasticity, like a rubber, under a room temperature.

Incidentally, the super-elastic alloy may be a nickel-titanium alloy. The treating instrument may further comprise a forceps member which is disposed at a forward end of the flexible sheath so that the forceps member is driven to be opened/closed by the operation wires.

The present disclosure relates to the subject matter contained in Japanese patent application No. 2000-152525 (filed on May 24, 2000), which is expressly incorporated herein by reference in its entirety.

### BRIEF DESCRIPTION OF THE DRAWINGS

FIG. 1 is a side sectional view of a forward end portion of an endoscopic biopsy forceps according to an embodiment of the present invention.

FIG. 2 is an overall configuration view of the endoscopic biopsy forceps according to the embodiment of the present invention.

### DESCRIPTION OF THE PREFERRED EMBODIMENT

An embodiment of the present invention will be described below with reference to the drawings.

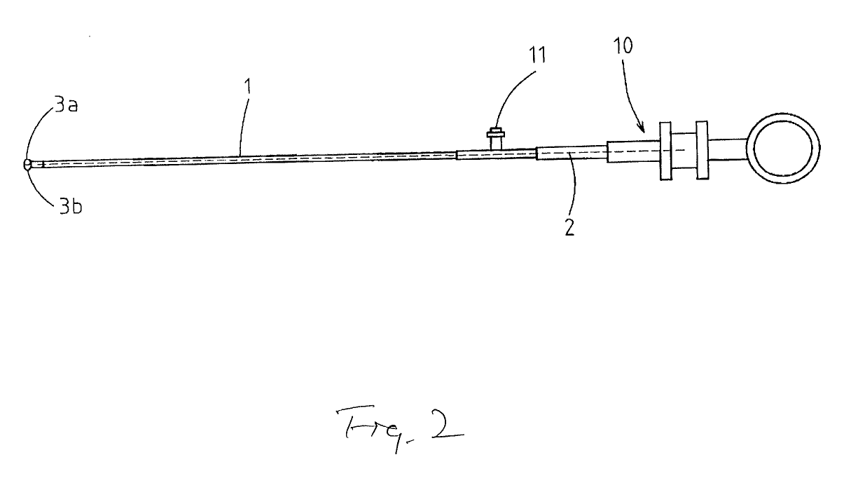

FIG. 2 shows a biopsy forceps for an endoscope for use in insertion into a treating instrument-pass channel of the endoscope. A flexible sheath 1 is formed by a pipe of a super-elastic alloy such as a nickel-titanium alloy.

Operation wires 2 are inserted and disposed in the full length of the flexible sheath 1 to move back and forth in the axial direction. A pair of forceps pieces 3a and 3b disposed on the forward end side of the flexible sheath 1 are driven to be opened/closed by the operation wires 2 moved back and forth axially from the handling side.

An operation portion 10 for operating the operation wires 2 to move back and forth is connected to the handling side end portion (base end portion) of the flexible sheath 1. An injection mouthpiece 11, for example, shaped like a Luer conical lock socket, for injecting liquid into the flexible sheath 1 is provided near the connected portion between the flexible sheath 1 and the operation portion 10.

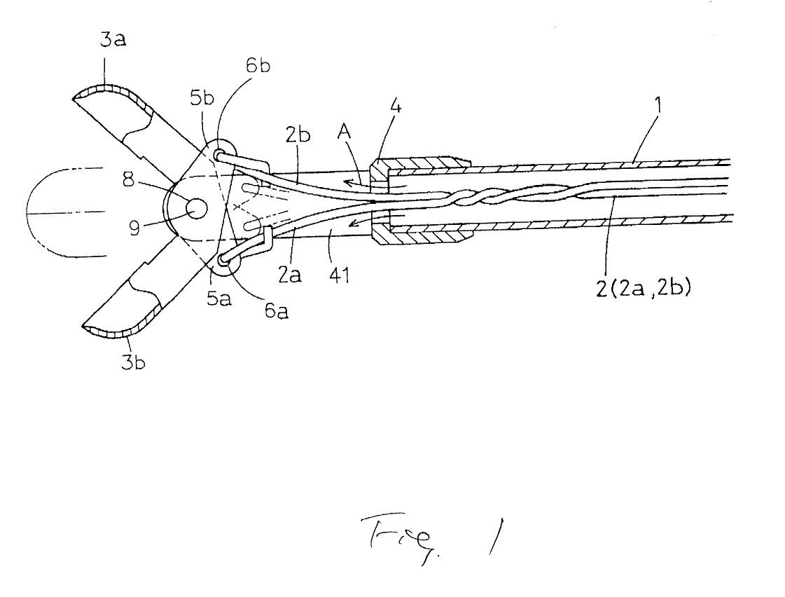

FIG. 1 shows the forward end portion of the flexible sheath 1 where the pair of forceps pieces 3a and 3b are disposed. A forward end support frame 4 is fixedly connected to the forward end of the flexible sheath 1 and the forward half portion of the frame 4 is largely divided like a fork by a slit 41.

Driving levers 5a and 5b are formed so as to be integrated with the pair of forceps pieces 3a and 3b respectively. A pivot 9 attached to a neighbor of the forward end of the forward end support frame 4 so as to cross the slit 41 is made to pass a through hole 8 formed in a boundary portion between the forceps piece 3a and the driving lever 5a and between the forceps piece 3b and the driving lever 5b respectively. Hence, the pair of forceps pieces 3a and 3b are opened/closed like a bill around the pivot 9.

As the operation wires 2, two wires 2a and 2b are made of a twisted or single stainless steel wire respectively , and are disposed side by side in the flexible sheath 1. Forward ends of the two wires 2a and 2b protrude forward from the forward end of the flexible sheath 1 so as to be fitted into small holes 6a and 6b formed near backward ends of the driving levers 5a and 5b respectively.

According to the aforementioned configuration, when the operation wires 2 are operated to move back and forth by the operation portion 10, the driving levers 5a and 5b connected to the forward ends of the wires 2a and 2b rotate respectively about the pivot 9 so as to open/close the forceps pieces 3a and 3b like a beak.

In the biopsy forceps for an endoscope configured as described above, the flexible sheath 1 is made of a super-elastic alloy pipe. Hence, the flexible sheath is sufficiently firm not to move unsteadily even in the case where the flexible sheath 1 has a small thickness (for example, in a range of from 0.1 to 0.2 mm). Moreover, the flexible sheath 1 hardly gets into the habit of bending because the flexible sheath 1 has a very large elastic limit.

The distortion factor of a nickel-titanium super-elastic alloy is from about 5 to about 8%. When, for example, the outer diameter of the flexible sheath 1 is about 2 mm, the flexible sheath 1 can be restored to its original straight state if external force is eliminated even in the case where the flexible sheath 1 is bent with a curvature radius of about 10 mm.

Moreover, the whole body of the flexible sheath 1 can be made of a smooth pipe free from roughness. Hence, dirt is hardly deposited on the flexible sheath 1. Even in the case where dirt is deposited on the flexible sheath 1, the flexible sheath 1 can be easily cleaned with a cleaning solution poured into the flexible sheath 1 through the injection mouthpiece 11 and discharged from the forward side of the flexible sheath 1 as represented by the arrow A in FIG. 1.

Moreover, the flexible sheath 1 can be made very thin. Hence, a space for pouring the cleaning solution can be secured in the inside of the flexible sheath 1. At the same time, the outer diameter of the flexible sheath 1 can be made small. Hence, inserting characteristic can be improved.

Incidentally, the present invention is not limited to the aforementioned embodiment. For example, the mechanism for opening/closing the forceps pieces 3a and 3b may be a pantagraph-like link mechanism, or the like. The present invention can be widely applied to various endoscopic treating instruments, such as various endoscopic forceps including endoscopic holding forceps and clipping forceps, or endoscopic holding instruments, measuring instruments, etc., which has the configuration that operation wires are inserted and disposed in a flexible sheath.

According to the present invention, the flexible sheath is made of a super-elastic alloy pipe. Hence, it has so excellent effects that the outer diameter of the flexible sheath can be made small, that the flexible sheath hardly gets into the habit of bending, that dirt is hardly deposited on the flexible sheath, and that the flexible sheath can be cleaned easily.

What is claimed is:

1. A treating instrument for an endoscope comprising a flexible sheath, and operation wires inserted and disposed in said flexible sheath so as to be movable back and forth axially, wherein said flexible sheath is formed by a pipe made of a super-elastic alloy.

2. A treating instrument for an endoscope according to claim 1, wherein said super-elastic alloy is a nickel-titanium alloy.

3. A treating instrument for an endoscope according to claim 1, further comprising a forceps member disposed at a forward end of said flexible sheath so that said forceps member is driven to be opened/closed by said operation wires.
